(19) United States
(12) Patent Application Publication
(10) Pub. No.: US 2006/0084694 A1
(43) Pub. Date: Apr. 20, 2006

(54) Ligand-dependant activation of Nur77 and uses thereof

(76) Inventors: Stephen H. Safe, College Station, TX (US); Sudhakar Chintharlapalli, College Station, TX (US)

Correspondence Address: HOWREY LLP;C/O IP DOCKETING DEPT., 2941 FAIRVIEW PARK DRIVE SUITE 200, FALLS CHURCH, VA, 22042-2924, US

(21) Appl. No.: 11/210,265

(22) Filed: Aug. 23, 2005

Related U.S. Application Data
(60) Provisional application No. 60/603,786, filed on Aug. 23, 2004.

(51) Int. Cl. A61K 31/404

(52) U.S. Cl. 514/414

(57) ABSTRACT

The present invention provides a method for inducing ligand-dependant Nur77 activation in a cell by contacting the cell with an effective dose of an agonist of Nur77. Also provided is a method for treating a mammal having a disease affected by modulation of Nur77 activity as well as a method for inducing apoptosis in a cell. The Nur77 agonists are preferably diindolylmethanes, and more preferably, methlylene-substituted diindolylmethanes. A representative example of the Nur77 agonists is a 1,1-bis (3′-indolyl)-1-(p-substituted phenyl) methane.

This application claims the benefit under 35 U.S.C. 119(e) of U.S. Provisional Application Ser. No. 60/603,786, filed Aug. 23, 2004, the entire contents of which are incorporated herein by reference.

### STATEMENT REGARDING FEDERALLY SPONSORED RESEARCH OR DEVELOPMENT

The government may own rights in the present invention pursuant to grant number ES09106 and CA108718 from the National Institute of Health.

### FIELD OF THE INVENTION

The field of the invention generally includes molecular biology and medical treatment. The invention relates to the ligand-dependant activation of Nur77 and applications of Nur77 activation. Particularly, the invention relates to a method for treating a disease affected by modulation of Nur77 activity, a method for activating Nur77 and a method for inducing apoptosis in a cell by using an agonist of Nur77.

### BACKGROUND OF THE INVENTION

The nuclear receptor superfamily of eukaryotic transcription factors encompasses steroid hormone and other nuclear receptors for which ligands have been identified and orphan receptors with no known ligands. (Tsai and O'Malley, 1994; Mangelsdorf, et al., 1995; Beato, et al., 1995; Olefsky, 2001; Enmark and Gustafsson, 1996; Giguere, 1999; Mohan and Heyman, 2003). Nuclear receptors share common structural features which include an N-terminal A/B domain, containing activation function-1 (AF-1), and a C-terminal E domain, which contains AF-2 and the ligand binding domain (LBD). Nuclear receptors also have a DNA binding domain (C domain), a variable hinge (D domain), and C-terminal F regions. Ligand activation of class 1 steroid hormone receptors induces homo- or heterodimer formation which interact with consensus or nonconsensus palindromic response elements. In contrast, class 2 receptors form heterodimers with the retinoic X receptor as a common partner, whereas class 3 and 4 orphan receptors act as homodimers or monomers and bind to direct response element repeats or single sites, respectively. The DNA binding domains of nuclear receptors all contain two zinc finger motifs that interact with similar half-site motifs; however, these interactions vary with the number of half-sites (1 or 2), their orientation, and spacing. Differences in nuclear receptor action are also determined by their other domains which dictate differences in ligand binding, receptor dimerization and interaction with other nuclear cofactors.

Most orphan receptors were initially cloned and identified as members of the nuclear receptor family based on their domain structure and endogenous or exogenous ligands have subsequently been identified for many of these proteins. (Enmark and Gustafsson, 1996; Giguere, 1999; Mohan and Heyman, 2003). The nerve growth factor I-B (NGFI-B) family of orphan receptors were initially characterized as immediate early genes induced by nerve growth factor in PC12 cells and the three members of this family include NGFI-Bα (Nur77), NGFI-Bβ (Nurr1), NGFI-Bγ (Nor1). (Milbrandt, 1988; Ryseck, et al., 1989; Nakai, et al., 1990).

Nur77 plays an important role in thymocyte-negative selection and in T-cell receptor (TCR)-mediated apoptosis in thymocytes (Winoto, 1997; He, 2002), and overexpression of Nur77 in transgenic mice resulted in high levels of apoptosis in thymocytes (Cheng, et al., 1997; Calnan, et al., 1995). In cancer cells, several mechanisms for Nur77-mediated apoptosis have been described and differences between studies may be due to the apoptosis-inducing agent or cell line. (Li, et al., 2000; Lin, et al., 2004; Wu, et al., 2002; Holmes, et al., 2003; Holmes, et al., 2003; Wilson, et al., 2003; Mu and Chang, 2003). For example, the retinoid 6-[3-(1-adamantyl)-4-hydroxyphenyl]-2-naphthalene carboxylic acid (CD437) and 12-O-tetradecanoylphorbol-13-acetate (TPA) induce translocation of Nur77 from the nucleus to the mitochondria where Nur77 binds Bcl-2 to form a pro-apoptotic complex. (Li, et al., 2000; Lin, et al., 2004). In contrast, it has been suggested that TPA-induced Nur77 in LNCaP prostate cancer cells activates transcription of E2F1 which is also pro-apoptotic. (Mu and Chang, 2003). These studies are examples of ligand-independent pathways where Nur77 expression is induced and/or Nur77 protein undergoes intracellular translocation since ligands for this receptor have hitherto not been reported. There is a need for developing a method for ligand-dependant activation of Nur77.

Compounds and compositions of substituted indole-3-carbinols and diindolylmethane have been used for treating estrogen-dependent conditions. (U.S. Pat. No. 5,948,808). Chen, et al., (1996) has suggested that 2,3,7,8-tetrachlorodibenzo-p-dioxin (TCDD) induced CYP1A1-dependent ethoxyresorufin O-deethylase (EROD) activity in human breast cells, and co-treatment with TCDD plus different concentrations of I3C or diindolylmethane resulted in a significant decrease in the induced response at the highest concentration of I3C or diindolylmethane. It is considered that diindolylmethane represents a new class of relatively non-toxic AhR-based antiestrogens that inhibit E2-dependent tumor growth in rodents. (Chen, et al., 1998).

Analogs of diindolylmethane have also been studied for their applications in treating estrogen-dependent conditions. For example, methyl substituted diindolylmethanes inhibited estrogen induced breast cancer growth. (McDougal and Safe, 1998). Dihalo-substituted analogs of diindolylmethane significantly inhibited mammary tumor growth while no significant changes in organ weights or liver and kidney histopathology were observed. (McDougal, et al., 2000). Ramamoorthy, et al. (1998) suggests that diindolylmethanes and substituted diindolylmethanes inhibit estrogen-induced uterine activities and breast cancer cell growth.

Cancer is one of the leading causes of premature death in most developed countries. Presently, many cancer treatments lack effectiveness or display significant negative side effects. Thus, there exists a need for the development of new and more effective treatments of cancer.

### SUMMARY OF THE INVENTION

The present invention is directed to the ligand-dependent activation of Nur77 and applications of Nur77 activation. Particularly, the present invention is directed to a method for inducing the ligand-dependent activation of Nur77 in a cell. This method comprises contacting the cell with an effective dose of an agonist of Nur77.

The invention is also directed to a method of treating a disease affected by modulation of Nur77 activity in a mammal. This method comprises administering to the mammal an effective dose of an agonist of Nur77.

The invention is further directed to a method for inducing apoptosis in a cell. This method comprises contacting the cell with an effective dose of an agonist of Nur77.

### DESCRIPTION OF THE FIGURES

FIG. 1 shows Nur77 expression and activation in cancer cell lines. (A) Western blot analysis of Nur77, Nurr1, and Nor1 protein expression in 12 cancer cell lines. (B) Activation of Gal4-Nur77 in Panc-28 cells treated with 10 or 20 μM of the various C-substituted diindolylmethane (DIM) compounds. (C) Activation of NuRE in Panc-28 cells treated with 10 or 20 μM of the various C-substituted DIM compounds. (D) Nur77 activation by isomeric DIM-C-pPhOCH₃ compounds.

FIG. 2 shows characterization and interactions of C-substituted DIMs that activate and inhibit Nur77-mediated transactivation. (A) Activation of GAL4-Nur77(E/F)/pGAL4. (B) Effects of iNur77 on transactivation. (C) Nur77 antagonist activity of DIM-C-pPhOH. (D) Nur77 antagonist activity of DIM-C-mPhOH. (E) Nur77 antagonist activity of DIM-C-oPhOH.

FIG. 3 shows DNA binding of Nur77 and ligand-induced coactivator-Nur77 interactions. (A) Gel mobility shift assay. (B) GAL4-coactivator interactions with VP-Nur77(E/F) in Panc-28 cells treated with DIM-C-pPhCF3. (C) GAL4-coactivator interactions with VP-Nur77(E/F) in Panc-28 cells treated with DIM-C-pPhOCH3. (D) GAL4-coactivator interactions with VP-Nur77(E/F) in Panc-28 cells treated with DIM-C-Ph.

FIG. 4 shows nuclear localization of Nur77. (A) Panc-28 cells immunostained for Nur77 and treated with DMSO or 10 μM Nur77 agonists for 6 hr. (B) Nuclear localization of in subcellular fractions of Panc-28 cells.

FIG. 5 shows that Nur77 agonists decrease cell survival and induce apoptosis. (A) Cell survival in Panc-28 cells treated with different concentrations of C-substituted DIMs for 4 days. (B) Effects of Nur77 agonists on PARP cleavage in Panc-28 cells. (C) Annexin staining of Panc-28 cells treated with camptothecin (positive control) or DIM-C-pPhOCH₃ for 6 hr. (D) Induction of apoptosis in LNCaP, MiaPaCa-1 and MCF-7 cells. (E) Induction of apoptosis in Panc-28 cells.

FIG. 6 shows Nur77-dependent induction of TRAIL and PARP cleavage in Panc-28 cells. (A) Induction of TRAIL. (B) Induction of TRAIL mRNA. (C) Effects of caspase inhibitors. (D) Effects of iNur77 on TRAIL expression and PARP cleavage in Panc-28 cells. (E) Inhibition of induced PARP cleavage and TRAIL by DIM-C-pPhOH.

FIG. 7 shows inhibition of tumor growth by DIM-C-pPhOCH₃. (A) Tumor area measurement vs. days after tumor injection. (B) Tumor weight in control and treatment animals.

### DETAILED DESCRIPTION OF THE INVENTION

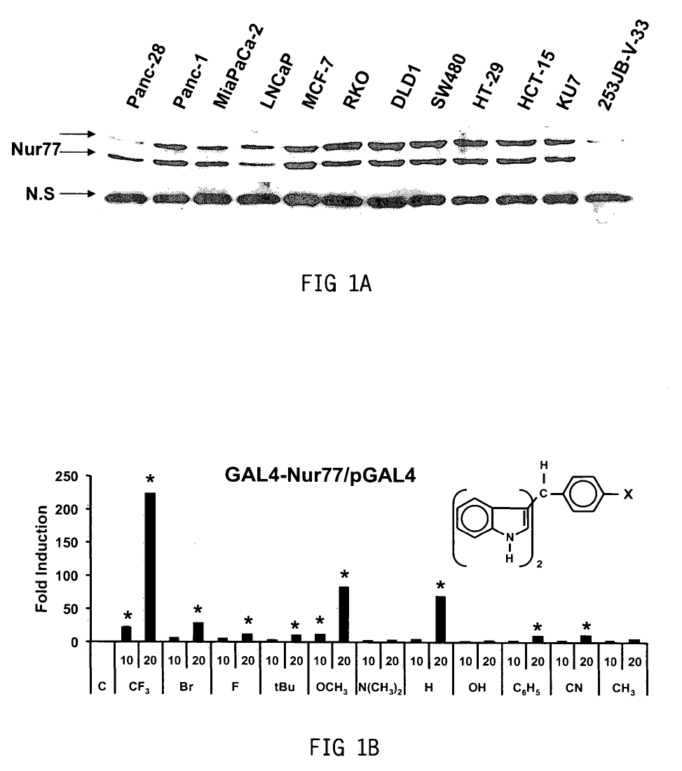

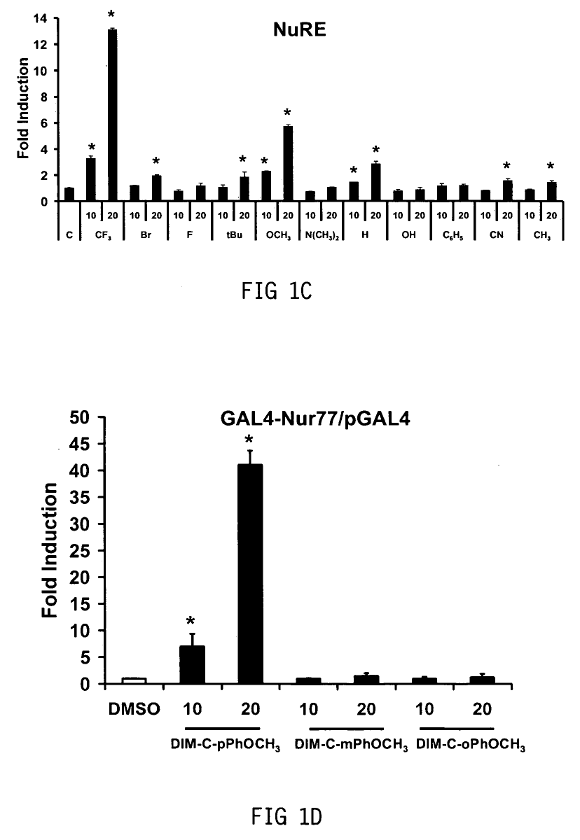

As illustrated in FIGS. 1-7, the present study demonstrates that 1,1-bis(3′-indolyl)-1-(p-substituted phenyl) methanes containing trifluoromethyl, hydrogen and methoxy substituents induce Nur77-dependent transactivation in Panc-28 pancreatic and other cancer cell lines. Also demonstrated is that Nur77 agonists induce typical cellular signatures of apoptosis including PARP cleavage and induction of TRAIL, and that both ligand-dependent transactivation and induction of apoptosis are associated with the action of nuclear Nur77. The present study shows for the first time that ligand-dependent activation of the orphan receptor Nur77 induces apoptosis in cancer cells, suggesting that Nur77 agonists represent a new class of anticancer drugs.

The present invention is directed to the ligand-dependent activation of Nur77 and applications of Nur77 activation. One aspect of the present invention provides a method for inducing the ligand-dependent activation of Nur77 in a cell. This method comprises contacting the cell with an effective dose of an agonist of Nur77. Preferably, the agonist of Nur77 is a diindolylmethane (DIM). More preferably, the agonist of Nur77 is a methlylene-substituted diindolylmethane.

Representative examples of preferred DIMs include 1,1-bis(3′-indolyl)-1-(p-substituted phenyl) methanes containing trifluoromethyl, hydrogen and methoxy substituents, i.e., DIMS having the chemical formula: 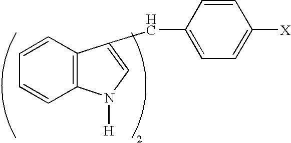
wherein X is H, trifluoromethyl, or methoxy.

Cells that are particularly amenable to ligand-dependant activation of Nur77 according to the present invention include cancer cells. According to one embodiment, cells are adrenal cortical cancer cells, anal cancer cells, bile duct cancer cells, bone cancer cells, bone metastasis cells, brain cancer cells, cervical cancer cells, non-Hodgkin's lymphoma cells, rectum cancer cells, esophageal cancer cells, eye cancer cells, gallbladder cancer cells, gastrointestinal carcinoid tumor cells, gestational trophoblastic disease cells, Hodgkin's disease cells, Kaposi's sarcoma cells, kidney cancer cells, laryngeal and hypopharyngeal cancer cells, leukemia cells, liver cancer cells, lung cancer cells, lung carcinoid tumors cells, malignant mesothelioma cells, metastatic cancer cells, multiple myeloma cells, myelodysplastic syndrome cells, nasal cavity and paranasal cancer cells, nasopharyngeal cancer cells, neuroblastoma cells, oral cavity and oropharyngeal cancer cells, osteosarcoma cells, ovarian cancer cells, pancreatic cancer cells, prostate cancer cells, breast cancer cells, colon cancer cells, and bladder cancer cells, penile cancer cells, pituitary cancer cells, retinoblastoma cells, salivary gland cancer cells, sarcoma cells, skin cancer cells, stomach cancer cells, testicular cancer cells, thymus cancer cells, thyroid cancer cells, uterine sarcoma cells, vaginal cancer cells, vulva cancer cells, or Wilm's tumor cells.

According to one embodiment, the cell is a human or a non-human mammalian cell, and can be in vivo or in vitro.

A further aspect of the present invention is a method for treating a mammal having a disease affected by modulation of Nur77 activity. This method comprises administering to the mammal an effective dose of an agonist of Nur77. Suitable agonists of Nur77 are DIMs and particularly suitable agonists are methlylene-substituted diindolylmethanes. Representative examples of suitable Nur77 agonists include 1,1-bis(3′-indolyl)-1-(p-substituted phenyl) methanes containing trifluoromethyl, hydrogen and methoxy substituents, i.e., DIMS having the chemical formula: 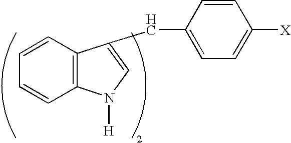
wherein X is H, trifluoromethyl, or methoxy.

Diseases amenable to treatment using the method of the present invention include the cancers described above, particularly pancreatic, prostate, breast, colon, and bladder cancer. Nur77 agonists can also be used for treating non-cancerous conditions such as a cardiovascular condition.

The method of the present invention can be used to treat diseases in humans or in a non-human mammal such as a mouse, rat, pig, cow, horse, dog, cat, monkey, rabbit, monkey, or sheep.

A still further aspect of the present invention is a method for inducing apoptosis in a cell. This method comprises contacting the cell with an effective dose of an agonist of Nur77. Preferably, the agonist of Nur77 is a diindolylmethane (DIM). More preferably, the agonist of Nur77 is a methlylene-substituted diindolylmethane.

Representative examples of preferred DIMs include 1,1-bis(3′-indolyl)-1-(p-substituted phenyl) methanes containing trifluoromethyl, hydrogen and methoxy substituents, i.e., DIMS having the chemical formula: 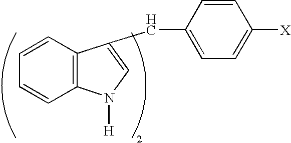
wherein X is H, trifluoromethyl, or methoxy.

Cells that are particularly suitable for the present method include cancer cells. According to one embodiment, cells are adrenal cortical cancer cells, anal cancer cells, bile duct cancer cells, bone cancer cells, bone metastasis cells, brain cancer cells, cervical cancer cells, non-Hodgkin's lymphoma cells, rectum cancer cells, esophageal cancer cells, eye cancer cells, gallbladder cancer cells, gastrointestinal carcinoid tumor cells, gestational trophoblastic disease cells, Hodgkin's disease cells, Kaposi's sarcoma cells, kidney cancer cells, laryngeal and hypopharyngeal cancer cells, leukemia cells, liver cancer cells, lung cancer cells, lung carcinoid tumors cells, malignant mesothelioma cells, metastatic cancer cells, multiple myeloma cells, myelodysplastic syndrome cells, nasal cavity and paranasal cancer cells, nasopharyngeal cancer cells, neuroblastoma cells, oral cavity and oropharyngeal cancer cells, osteosarcoma cells, ovarian cancer cells, pancreatic cancer cells, prostate cancer cells, breast cancer cells, colon cancer cells, and bladder cancer cells, penile cancer cells, pituitary cancer cells, retinoblastoma cells, salivary gland cancer cells, sarcoma cells, skin cancer cells, stomach cancer cells, testicular cancer cells, thymus cancer cells, thyroid cancer cells, uterine sarcoma cells, vaginal cancer cells, vulva cancer cells, or Wilm's tumor cells.

According to one embodiment, the cell is a human or a non-human mammalian cell, and can be in vivo or in vitro.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention. One of skill in the art will appreciate that certain embodiments of the invention provide methods of treating disease in humans and non-human mammals. These methods include all techniques common in the art for administering substances to mammals. It is within the skill of one in the art to optimize delivery techniques and dosages, depending on particular circumstances.

### EXAMPLES

### Example 1

### Nur77 Expression and Activation in Cancer Cell Lines

Panc-28, Panc-1, MiaPaCa-2, LNCaP, MCF-7, HT-29 and HCT-15 cancer cell lines were obtained from the American Type Culture Collection (Manassas, Va.). RKO, DLD-1 and SW-480 colon cancer cells were provided by Dr. S. Hamilton, and KU7 and 253-JB-V-33 bladder cells were provided by Dr. A. Kamat (M.D. Anderson Cancer Center, Houston, Tex.).

The C-substituted DIMs were synthesized as previously described (Qin, et al., 2004). Antibodies for PARP (sc8007), Sp1 (sc-59) and TRAIL (sc7877) were purchased from Santa Cruz Biotechnology (Santa Cruz, Calif.) and Nur77 (IMG-528) from Imgenex (San Diego, Calif.).

The GAL 4 reporter containing five GAL4 response elements (pGAL4) was provided by Dr. Marty Mayo (University of North Carolina, Chapel Hill, N.C.). The GAL4-Nur77 (full length) and GAL4-Nur77 (E/F) chimeras were provided by Dr. Jae W. Lee (Baylor College of Medicine, Houston, Tex.) and Dr. T. Perlmann (Ludwig Institute for Cancer Research, Stockholm, Sweden) respectively, and Dr. Lee also provided the Nur77 response element-luciferase (NurRE-Luc) reporter construct. The GAL-4-coactivator fusion plasmids pM-SRC1, pMSRC2, pMSRC3, pM-DRIP205 and pMCARM-1 were kindly provided by Dr. Shigeaki Kato (University of Tokyo, Tokyo, Japan). A non-specific scrambled (iScr) oligonucleotide as described (Abdelrahim, et al., 2002) was used for RNA interference assays.

The small inhibitory RNA for Nur77 (iNur77) was identical to the reported oligonucleotide (Lin, et al., 2004) and these were purchased from Dharmacon Research (Lafayette, Colo.). Leptomycin B (LMB) was obtained from Sigma (St Louis, Mo.) and caspase inhibitors were purchased from BD Pharminogen (San Diego, Calif.).

The following oligonucleotides were prepared by IDT (Coralville, Iowa) and were used in gel mobility shift assays; NBRE:5′-GAT CCT CGT GCG AAA AGG TCA AGC GCT A-3′; NurRE:5′-GAT CCT AGT GAT ATT TAC CTC CAA ATG CCA GGA-3′.

Whole cell lysates from 12 different cancer cell lines derived from pancreatic, prostate, breast, colon and bladder tumors were analyzed for Nur77, Nurr1 and Nor1 by Western blot analysis. FIG. 1A summarizes the result of the Western blot analysis. The results show that only the 253 JB-V-33 bladder cancer cell line exhibited relatively low expression of Nur77, and the antibodies and electrophoretic conditions gave two immunostained bands as previously reported in other studies. Western blot analysis of the other NGFI-B proteins showed variable expression of Nurr1, and Nor1 was not detectable in these cancer cell lines (data not shown). Similar results were also obtained in Jurkat T-cell leukemia cells (data not shown).

Structure-dependent activation of Nur77 by a series of eleven C-substituted DIMs was investigated in Panc-28 cells transfected with a GAL4-Nur77 (full length) chimera and a reporter construct containing five GAL4 response elements linked to a luciferase reporter gene (pGAL4). FIG. 1B shows that three compounds containing p-trifluoromethyl (DIM-C-pPhCF₃) and methoxy (DIM-C-pPhOCH₃) substituents or the unsubstituted phenyl group (DIM-C-Ph) activated luciferase activity. Similar results were also obtained in Panc-28 cells transfected with a construct containing a Nur response element (NurRE) (FIG. 1C), and these same compounds also activated GAL4-Nur77/pGAL4 and NurRE in MiaPaCa-1 pancreatic, HCT-15 colon, and MCF-7 breast cancer cells (data not shown).

The structure-dependent activation of Nur77 was also investigated using DIM-C-pPhOCH₃ as a model and the position of the methoxyl group was changed to the meta (DIM-C-mPhOCH₃) and ortho (DIM-C-oPhOCH₃) positions. (FIG. 1D). Only the para-substituted compound was active.

N-methyl and 2-methyl indole ring-substituted analogs of DIM-C-pPhOCH₃, DIM-C-Ph, and DIM-C-pPhCF₃ were also investigated. These compounds did not activate Nur77 (data not shown). These results demonstrate that activation of Nur77 by C-DIMs was structure-dependent and sensitive to substitution on the phenyl and indole rings. Thus, at least three C-substituted DIMs activate Nur77; one of these compounds (DIM-C-pPhCF₃) also activates PPARγ (Qin, et al., 2004; Chintharlapalli, et al., 2004), whereas DIM-C-pPhOCH₃ and DIM-C-Ph are PPARγ-inactive (Qin, et al., 2004). DIM-C-pPhOH was inactive in both transactivation assays and, at higher concentrations, decreased activity lower than observed in solvent (DMSO) control.

### EXAMPLE 2

### Characterization and Interactions of C-DIMs that Activate and Inhibit Nur77-Mediated Transactivation

Transfection assays were essentially carried out as previously described using Lipofectamine Plus reagent (Invitrogen, Carlsbad, Calif.) and luciferase activities were normalized to β-galactosidase activity. For RNA interference studies, cells were transfected with small inhibitor RNAs for 36 hr to ensure protein knockdown prior to the standard transfection and treatment protocols (Qin, et al., 2004; Abdelrahim, et al., 2002). Results are expressed as means±SE for at least three replicate determinations for each treatment group.

Panc-28 cells were plated in 12-well plates at 1×10⁵ cells/well in DME-F12 media supplemented with 2.5% charcoal-stripped FBS. After growth for 16 hr, various amounts of DNA, i.e. Gal4Luc (0.4 μg), β-gal (0.04 μg), VP-Nur77(E/F) (0.04 μg), pM SRC1 (0.04 μg), pMSRC2 (0.04 μg), pMSRC3 (0.04 μg), pMDRIP205 (0.04 μg) and pMCARM-1 (0.04 μg) were transfected by Lipofectamine (Invitrogen) according to the manufacturer's protocol. After 5 hr of transfection, the transfection mix was replaced with complete media containing either vehicle (DMSO) or the indicated ligand for 20-22 hr. Cells were then lysed with 100 ml of 1X reporter lysis buffer, and 30 μl of cell extract were used for luciferase and β-Gal assays. Lumicount was used to quantitate luciferase and β-Gal activities, and the luciferase activities were normalized to β-Gal activity.

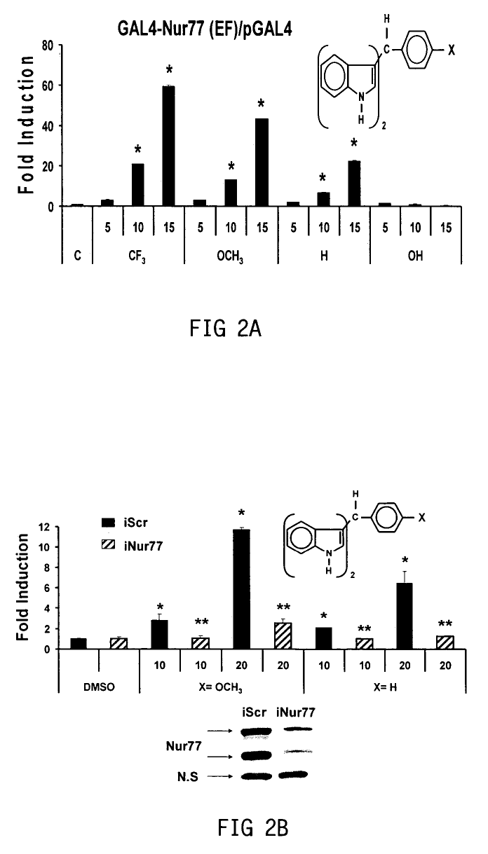

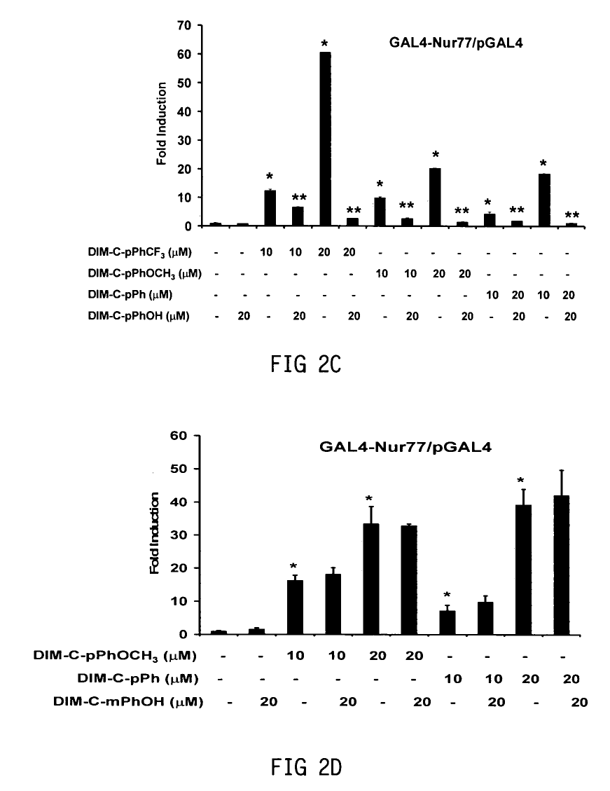

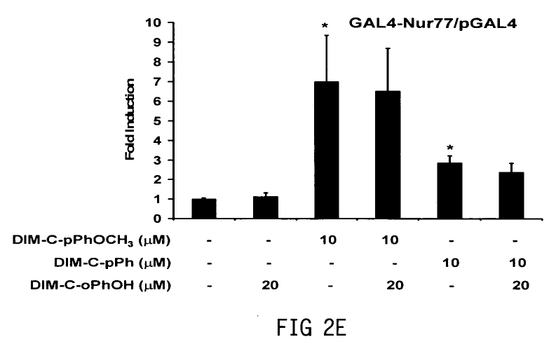

The role of the LBD or E/F region in ligand-induced transactivation of Nur77 was investigated in Panc-28 cells transfected with pGAL4 and a chimeric GAL4-Nur77(E/F) construct containing only the E/F domain of Nur77. Treatment of Panc-28 cells with different concentrations (5-15 μM) of DIM-C-pPhCF3, DIM-C-pPhOCH3 and DIM-C-Ph induced luciferase activity, whereas no response was observed in cells treated with Nur77-inactive DIM-C-pPhOH. (FIG. 2A). These results are the first to identify a series of compounds that directly activate Nur77(LBD)-dependent transactivation in Panc-28 or any other cancer cell line. The role of Nur77 in mediating transactivation was further investigated in Panc-28 cells treated with 10 or 20 μM DIM-C-pPhOCH3 or DIM-C-Ph and transfected with pNurRE, a non-specific “scrambled” small inhibitory RNA (iScr), or small inhibitory RNA for Nur77 (iNur77). The results (FIG. 2B) showed decreased Nur77 protein in whole cell lysates and a 90-100% decrease in ligand-induced transactivation over the different concentrations of compounds, thus confirming the role of Nur77 in mediating this response. As noted above, one compound which contained a p-hydroxy substituent (DIM-C-pPhOH) did not induce activity (FIG. 1B) and DIM-C-pPhOH was further investigated as a potential Nur77 antagonist. Panc-28 cells were transfected with GAL4-Nur77/pGAL4 and cotreated with DIM-C-pPhOH and Nur77 agonists DIM-C-pPhCF3, DIM-C-pPhOCH3, and DIM-C-pH (FIG. 2C). The results show that DIM-C-pPhOH antagonizes activation of Nur77 by all three C-DIM compounds. The structural specificity of Nur77 antagonists was further investigated using meta-hydroxy (DIM-C-mPhOH) and ortho-hydroxy (DIM-C-oPhOH) analogs. DIM-C-mPhOH (10 or 20 μM) did not inhibit DIM-C-pPhOCH3- or DIM-C-Ph-induced transactivation (FIG. 2D). DIM-C-oPhOH also did not exhibit Nur77 antagonist activity (FIG. 2E); however, high doses (20 μM) of both Nur77 agonists and DIM-C-oPhOH were toxic. Thus, activation of Nur77 by C-DIMs was E/F domain-dependent and Nur77 activation was inhibited by DIM-C-pPhOH; moreover, both activation and inhibition of Nur77-mediated transactivation was dependent on the structure of the C-DIM compounds.

### EXAMPLE 3

### Nur77 DNA-binding and C-DIM-induced Nur77-coactivator Interactions

Cells were seeded in DME/F12 medium supplemented with 2.5% charcoal-stripped serum and treated with 10 μM DIM-C-pPhOCH₃ for 30 min. Nuclear extracts were obtained using NE-PER nuclear and cytoplasmic extraction reagents (Pierce Chemical Co.). Oligonucleotides were synthesized, purified, and annealed, and 5 pmol of specific oligonucleotides were ³²P-labeled at the 5′-end using T₄ polynucleotide kinase and [³²γP]ATP. Nuclear extracts were incubated in HEPES with ZnCl₂ and 1 μg poly deoxyinosine-deoxycytidine for 5 min; 100-fold excess of unlabeled wild-type or mutant oligonucleotides were added for competition experiments and incubated for 5 min. The mixture was incubated with labeled DNA probe for 15 min on ice. The reaction mixture was loaded onto a 5% polyacrylamide gel and ran at 150 V for 2 hr. The gel was dried and protein DNA complexes were visualized by autoradiography using a Molecular Dynamics, Inc. Storm 860 instrument (Amersham Biosciences).

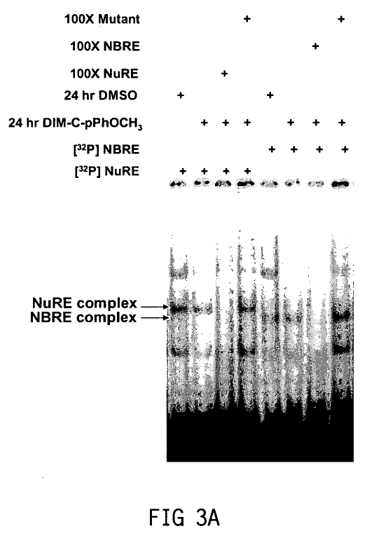

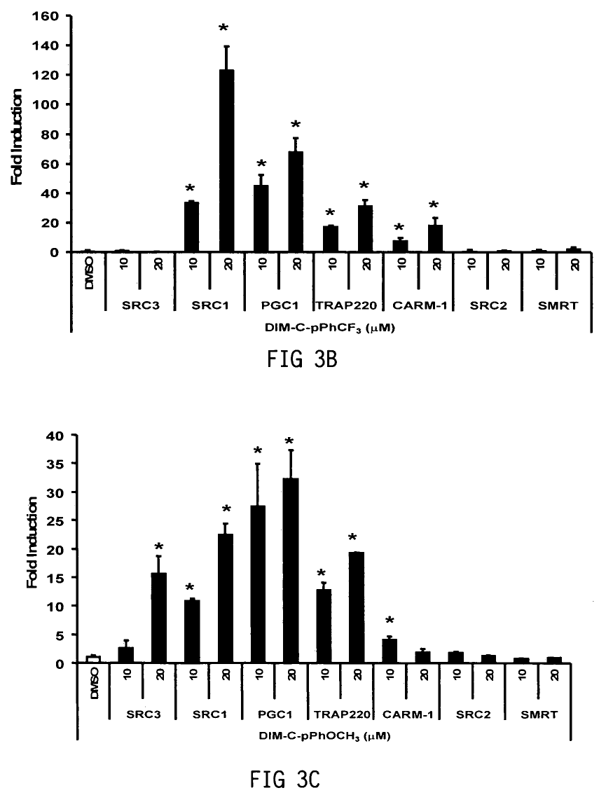

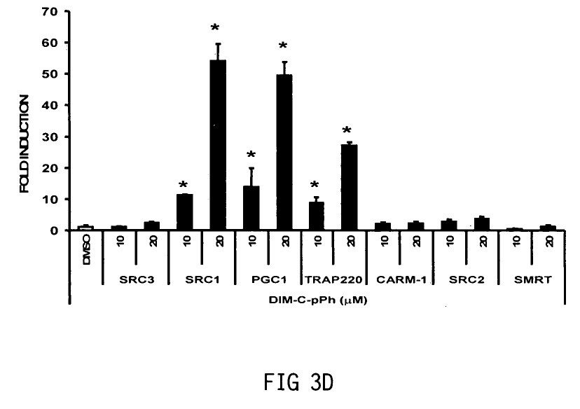

Incubation of nuclear extracts from Panc-28 cells treated with DMSO or DIM-C-pPhOCH₃ with ³²P-labeled NBRE and NurRE (lanes 1,2 and 5,6, respectively) gave retarded bands in EMSA assays (FIG. 3A). Retarded band intensities were decreased after incubation with 100-fold excess NurRE (lane 3) or NBRE (lane 7) but not by mutant NurRE (lane 4) or mutant NBRE (lane 8) oligonucleotides. These results show that nuclear extracts containing Nur77 bind NurRE and NBRE as dimers and monomers, respectively, and this corresponds to their migration in the EMSA assay. Results obtained for nuclear extracts from solvent-treated cells show that formation of the retarded bands is ligand-independent and the retarded band pattern corresponds to previous studies using nuclear extracts from cells or in vitro translated Nur77 (Philips, et al., 1997; Maira, et al., 2003). Extracts from cells treated with Nur77-active C-substituted DIMs gave retarded band intensities similar to that observed for solvent-treated extracts suggesting minimal ligand-dependent loss of nuclear Nur77 in these cells.

Ligand-dependent activation of nuclear receptors is dependent on interaction of the bound receptor with coactivators (Rosenfeld and Glass, 2001; Xu and Li, 2003; Smith and O'Malley, 2004) and FIGS. 3B-3D summarize results of a mammalian two-hybrid assay in Panc-28 cells transfected with VP-Nur77 (ligand binding domain) and GAL4-coactivator chimeras. Ligand-induced Nur77-coactivator interactions were determined using a construct (pGAL4) containing 5 GAL4 response elements. Coactivators used in this study include SRC-1, SRC-2 (TIFII), SRC-3 (AIB1), PGC-1, TRAP220 and CARM-1. A GAL4-repressor (SMRT) chimera was also included in the assay. All three ligands induced transactivation in cells transfected with GAL4-SRC-1, GAL4-PGC-1 and GAL4-TRAP220 chimeras. DIM-C-pPhOCH₃ induced transactivation in cells transfected with GAL4-SRC-3 and GAL4-CARM-1 was slightly activated by DIM-C-pPhOCH₃ and DIM-C-pPhCF₃. The results demonstrate that although there were some ligand-dependent differences in transactivation observed for GAL4-SRC-3 and GAL4-CARM-1; however, the most significant interactions between VP-Nur77 and GAL4 chimeras expressing SRC-1, PGC-1 and TRAP220 were induced by all three compounds.

### EXAMPLE 4

### Effects of Nur77-active C-DIMs on Cell Survival and Apoptosis and Role of Nuclear Nur77

The different cancer cell lines were cultured under standardized conditions. Panc-28 cells were grown in DMEM:Ham's F-12 media containing 2.5% charcoal stripped fetal bovine serum, and cells were treated with DMSO and different concentrations of test compounds as indicated. For longer term cell survival studies, the media was changed every second day, and values were presented for a 4 day experiment. For all other assays, cytosolic, nuclear fractions, or whole cell lysates were obtained at various time points, analyzed by Western blot analysis, and bands were quantitated as previously described (Qin, et al., 2004; Abdelrahim, et al., 2002). Immunocytochemical analysis was determined using Nur77 antibodies as previously reported (Abdelrahim, et al., 2002).

Detection of phosphatidylserine on the outside of the cell membrane, a unique and early marker for apoptosis, was performed using a commercial kit (Vybrant Apoptosis Assay Kit #2; Molecular Probes, Eugene, Oreg.). Panc-28 cells were cultured as described above, and treated with 10 μM DIM-C-pPhOCH₃ or camptothecin for 6, 12 and 24 hrs. Binding of annexin V-Alexa-488 conjugate and propidium iodide (PI) was performed according to the manufacturer's instructions. After binding and washing, cells were observed under phase contrast and epifluorescent illumination using a 495-nm excitation filter and a 520-nm absorption filter for annexin V-Alexa 488 and a 546-nm excitation filter and a 590-nm absorption filter for PI. Healthy cells were unstained by either dye; cells in early stages of apoptosis were stained only by annexin V, while dead cells were stained by annexin V and PI. The assay was repeated on three separate Panc-28 cell preparations.

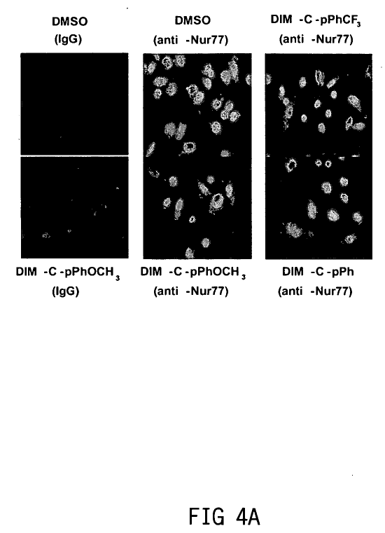

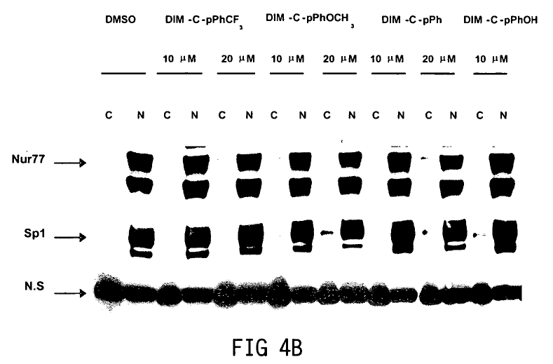

In several cancer cell lines transfected with Nur77-GFP constructs, treatment with apoptosis and differentiation-inducing agents results in rapid translocation of Nur77 into the cytosol/mitochondria (Li, et al., 2000; Lin, et al., 2004; Wu, et al., 2002; Holmes, et al., 2003; Holmes, et al., 2003; Wilson, et al., 2003). Similar results have been observed in BGC-823 human gastric cancer cells where endogenous Nur77 is nuclear and TPA induced Nur77 translocation into the cytosol and this was accompanied by apoptosis but not by Nur77-dependent transactivation (Wu, et al., 2003). Results summarized in FIG. 4A show immunostaining of Nur77 in the nucleus of Panc-28 cells treated with DMSO and Nur77-active DIM-C-pPhCF₃, DIM-C-pPhOCH₃ and DIM-C-Ph for 6 hr, and comparable results were obtained in Panc-28, MiaPaCa and LNCaP cells after treatment for 6 or 12 hr (data not shown). In all cases, Nur77 remained in the nucleus, and cells exhibited a compacted nuclear staining pattern typically observed in cells activated for cell death pathways. In a separate experiment, Panc-28 cells were treated with 10 or 20 μM DIM-C-pPhCF₃, DIM-C-pPhOCH₃ and DIM-C-Ph or 10 μM DIM-C-pPhOH for 12 hr, and Nur77 protein levels were determined by Western blot analysis of cytosolic and nuclear extracts. (FIG. 4B). These results also confirm that Nur77, in the presence or absence of C-substituted DIM agonists, is a nuclear protein and ligand-induced Nur77 translocation from the nucleus is not observed. Sp1 is a nuclear protein and was used as a control to ensure efficient separation of the two extracts and Sp1 was identified only in the nuclear fraction. (FIG. 4B).

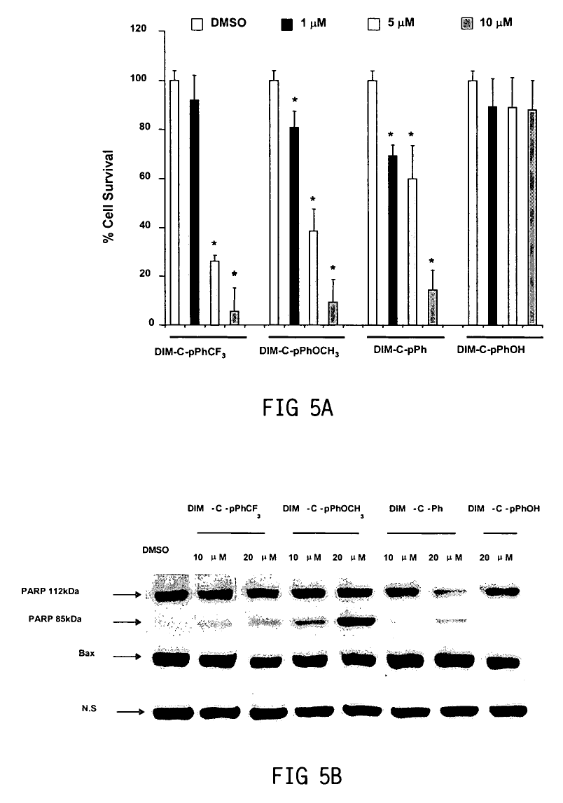

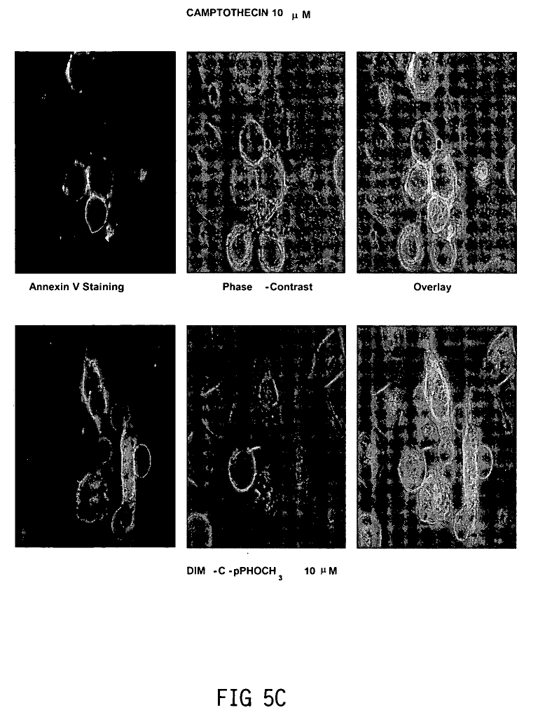

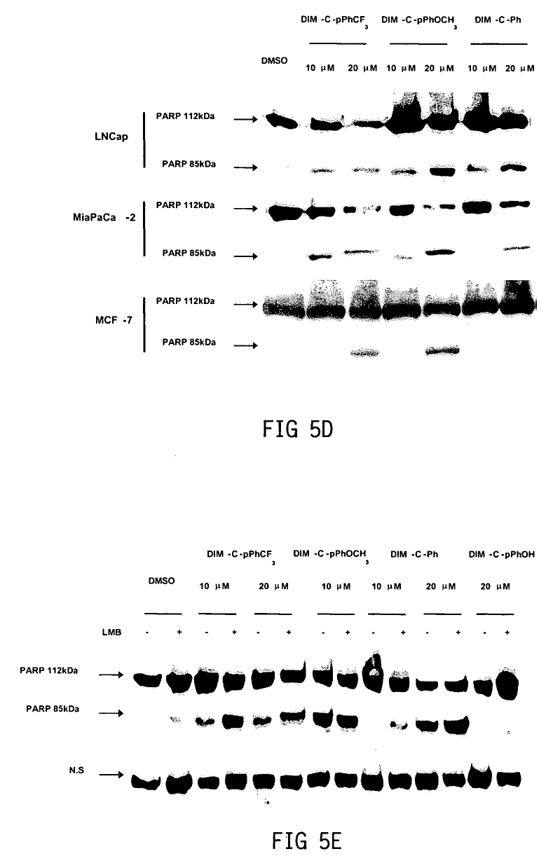

Nur77 agonists significantly decreased survival of Panc-28 cells (FIG. 5A), and IC₅₀ values for DIM-C-pPhCF₃, DIM-C-pPhOCH₃ and DIM-C-Ph were between 1-5 μM, whereas DIM-C-pPhOH did not affect cell survival. At longer time points (4 and 6 days), DIM-C-pPhOH slightly inhibited cell proliferation; however, induction of cell death was not observed for this compound at concentrations as high as 20 μM. Decreased cell survival is also observed for agents that induce apoptosis and/or Nur77 nuclear to cytosolic translocation in cancer cells (Li, et al., 2000; Lin, et al., 2004; Wu, et al., 2002; Holmes, et al., 2003; Holmes, et al., 2003; Wilson, et al., 2003). Results illustrated in FIG. 5B show that treatment of Panc-28 cells with Nur77 agonists induced cleavage of PARP, whereas the Nur77-inactive DIM-C-pPhOH did not induce this response. PARP cleavage is associated with activation of cell death pathways; however, this was not accompanied by changes in levels of bax (FIG. 5B) or bcl-2 proteins (data not shown). Moreover, treatment of Panc-28 cells with 10 and 20 μM DIM-C-pPhOCH₃ for 8 and 12 hr showed a time- and dose-dependent increase of annexin V-stained cells using a green-fluorescent Alexa Fluor 488 probe (FIG. 5C). The effects of camptothecin (positive control for apoptosis) and DIM-C-pPhOCH₃ were comparable. After treatment with DIM-C-pPhOCH₃ for 6 hr, annexin V-stained cells were significantly increased, plasma membrane blebbing was observed, and there was minimal PI staining. However, after 12 hr, PI staining was increased. Induction of PARP cleavage by Nur77 agonists was also observed in other pancreatic (MiaPaCa-2), prostate (LNCaP) and breast (MCF-7) cancer cell lines (FIG. 5D). Induction of PARP cleavage by the Nur77-active compounds in Panc-28 cells was not accompanied by changes in Nur77 expression (FIG. 4B), and this was in contrast to TPA which activates nuclear pathways by inducing Nur77 expression (Mu and Chang, 2003). Using a protocol comparable to that outlined in FIG. 5B, the induction of PARP cleavage by the Nur77 agonists in Panc-28 cells was not affected by the nuclear export inhibitor leptomycin B (LMB) (1 ng/ml). (FIG. 5E). LMB alone slightly induced PARP cleavage and, for some cells cotreated with LMB plus Nur77 agonists, there was enhanced PARP cleavage. In contrast, previous studies showed that LMB inhibits apoptosis in cells treated with apoptosis-inducing agents that activate nuclear-cytosol/mitochondrial translocation of Nur77 (Li, et al., 2000; Lin, et al., 2004). These results demonstrate that activation of nuclear Nur77 by C-substituted DIMs induces apoptosis in Panc-28 and other cancer cell lines; however, evidence for activation of the intrinsic apoptotic pathways was not observed.

### EXAMPLE 5

### Nur77-active C-DIMs Induce TRAIL

cDNA was prepared from the Panc-28 cell line using a combination of oligodeoxythymidylic acid (Oligo d(T)₁₆), and dNTP mix (Applied Biosystems) and Superscript II (Invitrogen). Each PCR was carried out in triplicate in a 20-μl volume using Sybr Green Mastermix (Applied Biosystems) for 15 min at 95° C. for initial denaturing, followed by 40 cycles of 95° C. for 30 s and 60° C. for 1 min in the ABI Prism 7700 Sequence Detection System. The ABI Dissociation Curves software was used following a brief thermal protocol (95° C. 15 s and 60° C. 20 s, followed by a slow ramp to 95° C.) to control for multiple species in each PCR amplification. Values for each gene were normalized to expression levels of TBP. The sequences of the primers used for RT-PCR were as follows: TRAIL forward, 5′-CGT GTA CTT TAC CAA CGA GCT GA-3′, reverse, 5′-ACG GAG TTG CCA CTT GAC TTG-3′; and TBP forward, 5′-TGC ACA GGA GCC AAG AGT GAA-3′, reverse, 5′-CAC ATC ACA GCT CCC CAC CA-3′.

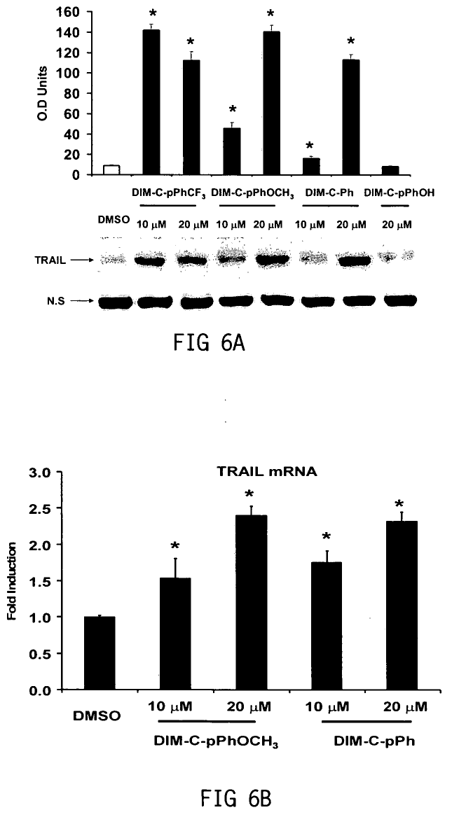

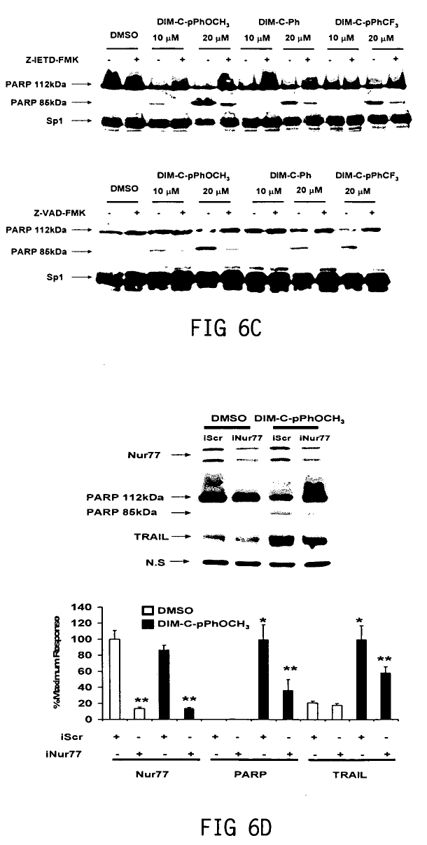

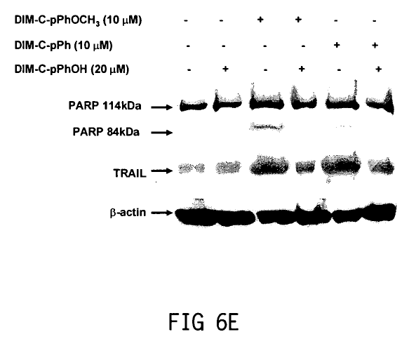

In thymocytes, there is evidence that Nur77-induced apoptosis is linked to transcriptional activation (uang, et al., 1999), and microarray studies in thymocytes undergoing Nur77-dependent apoptosis identified several apoptosis-related genes including fasL and TRAIL (Rajpal, et al., 2003). Results in FIG. 6A show that Nur77 agonists that induce PARP cleavage also induce TRAIL (but not fasL) protein expression in Panc-28 cells, suggesting that this response may be a direct or indirect downstream target of Nur77 agonists in cancer cells. The Nur77-inactive DIM-C-pPhOH did not induce TRAIL. In addition, DIM-C-pPhOCH₃ or DIM-C-Ph induced TRAIL mRNA levels in Panc-28 cells. (FIG. 6B). Since TRAIL activates the extrinsic apoptosis pathway and activation of caspase 8, the effect of a caspase 8 inhibitor (Z-IETD-FMK) and the pan-caspase inhibitor (Z-VAD-FMK) was also investigated on induction of PARP cleavage by Nur77 agonists (FIG. 6C). The results show that both inhibitors blocked (60-90%) induction of PARP cleavage by Nurr7 agonists.

The role of Nur77 in mediating induction of TRAIL and PARP cleavage by DIM-C-pPhOCH₃ was further investigated in Panc-28 cells transfected with non-specific RNA (iScr) and iNur77 (FIG. 6D). Levels of Nur77, PARP cleavage, and TRAIL proteins were determined by Western blot analysis of whole cell extracts and the results showed that iNurr significantly decreased levels of all three proteins. In addition, cotreatment of Panc-28 cells with DIM-C-pPhOH₃ or DIM-C-Ph and the Nur77 antagonist DIM-C-pPhOH (FIG. 6E) showed that the latter compound also inhibited induction of PARP cleavage and TRAIL protein expression induced by Nur77 agonists. These results demonstrate that Nur77 agonists induce apoptosis pathways in cancer cells through transcriptional (nuclear) mechanisms, and at least one of the induced proteins (TRAIL) activates an extrinsic apoptotic pathway. In summary, selected C-substituted DIMs have now been identified as ligands for the orphan receptor Nur77 and activation of this receptor is associated with decreased cancer cell survival, induction of TRAIL and apoptosis.

### EXAMPLE 6

### Inhibition of Tumor Growth in Athymic Nude Mice Bearing Panc-28 Cell Xenografts

Male athymic nude mice (BALB/c, ages 8-12 weeks) were purchased from Harlan (Indianapolis, Ind.). The mice were housed and maintained in laminar flow cabinets under specific pathogen-free conditions. Panc-28 cells were harvested from subconfluent cultures by trypsinization and washed. Panc-28 cells (2×10⁶) were injected subcutaneously into each mouse on both flanks using a 30-gauge needle. The tumors were allowed to grow for 11 days until tumors were palpable. Mice were then randomized into two groups of seven mice per group and dosed by oral gavage with either corn oil or DIM-C-pPhOCH₃ every second day. The volume of corn oil was 75 μl, and the dose of DIM-C-pPhOCH₃ was 25 mg/kg/day. The mice were weighed, and tumor areas were also measured ever other day. Final body and tumor weights were determined at the end of the dosing regiment; and selected tissues were further examined by routine H & E staining and immunohistochemical analysis for apoptosis using the TUNEL assay.

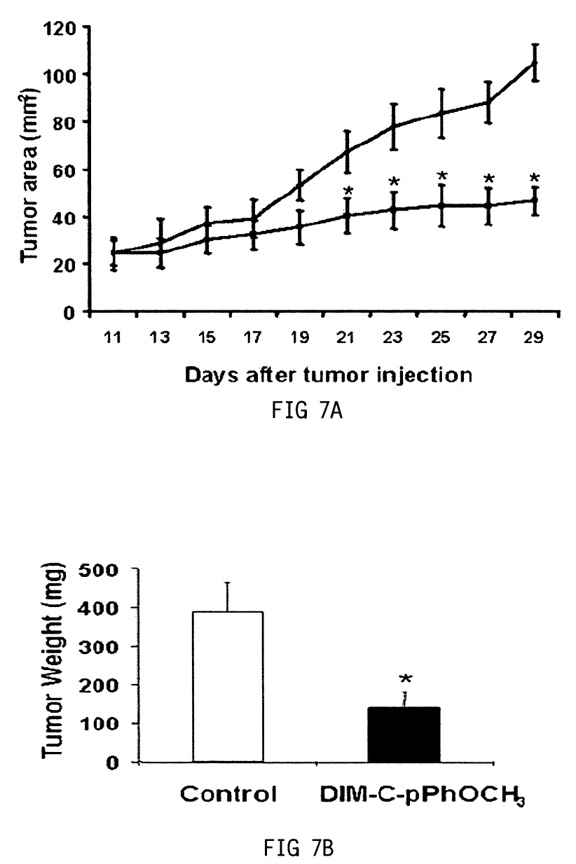

The results (FIG. 7A) showed that DIM-C-pPhOCH₃ significantly inhibited tumor growth (area), and this was also complemented by a parallel decrease in tumor weights (FIG. 7B). Analysis of tumors from control and treated animals (TUNEL assay) indicated similar levels of apoptosis. Animal weight gain and organ weights were comparable in both treatment groups, and there were no apparent signs of toxicity in the DIM-C-pPhOCH₃-treated mice compared with the corn oil controls. The mouse brain and muscle express relatively high levels of Nur77 (Law, et al., 1992), and examination of brain regions by H & E staining did not indicate any differences between the control (corn oil) and DIM-C-pPhOCH₃-treated animals.

What is claimed is:

1. A method for inducing ligand-dependent activation of Nur77 in a cell comprising contacting the cell with an effective dose of an agonist of Nur77.

2. The method of claim 1, wherein the agonist of Nur77 is a diindolylmethane.

3. The method of claim 2, wherein the agonist of Nur77 is a methlylene-substituted diindolylmethane.

4. The method of claim 3, wherein the agonist of Nur77 is a 1,1-bis (3′-indolyl)-1-(p-substituted phenyl) methane.

5. The method of claim 4, wherein the agonist of Nur77 has the chemical formula: 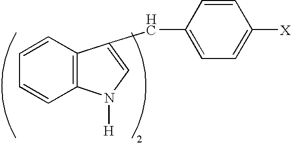
wherein X is H, trifluoromethyl, or methoxy.

6. The method of claim 1, wherein the cell is a cancer cell.

7. The method of claim 6, wherein the cancer cell is selected from the group consisting of adrenal cortical cancer cell, anal cancer cell, bile duct cancer cell, bone cancer cell, bone metastasis cell, brain cancer cell, cervical cancer cell, non-Hodgkin's lymphoma cell, rectum cancer cell, esophageal cancer cell, eye cancer cell, gallbladder cancer cell, gastrointestinal carcinoid tumor cell, gestational trophoblastic disease cell, Hodgkin's disease cell, Kaposi's sarcoma cell, kidney cancer cell, laryngeal and hypopharyngeal cancer cell, leukemia cell, liver cancer cell, lung cancer cell, lung carcinoid tumor cell, malignant mesothelioma cell, metastatic cancer cell, multiple myeloma cell, myelodysplastic syndrome cell, nasal cavity and paranasal cancer cell, nasopharyngeal cancer cell, neuroblastoma cell, oral cavity and oropharyngeal cancer cell, osteosarcoma cell, ovarian cancer cell, pancreatic cancer cell, prostate cancer cell, breast cancer cell, colon cancer cell, bladder cancer cell, penile cancer cell, pituitary cancer cell, retinoblastoma cell, salivary gland cancer cell, sarcoma cell, skin cancer cell, stomach cancer cell, testicular cancer cell, thymus cancer cell, thyroid cancer cell, uterine sarcoma cell, vaginal cancer cell, vulva cancer cell, and Wilm's tumor cell.

8. The method of claim 1, wherein the cell is a human or non-human mammalian cell.

9. The method of claim 8, wherein the cell is in vivo or in vitro.

10. A method for treating a mammal having a disease affected by modulation of Nur77 activity comprising administering to the mammal an effective dose of an agonist of Nur77.

11. The method of claim 10, wherein the agonist of Nur77 is a diindolylmethane.

12. The method of claim 11, wherein the agonist of Nur77 is a methlylene-substituted diindolylmethane.

13. The method of claim 12, wherein the agonist of Nur77 is a 1,1-bis (3′-indolyl)-1-(p-substituted phenyl) methane.

14. The method of claim 13, wherein the agonist of Nur77 has the chemical formula: 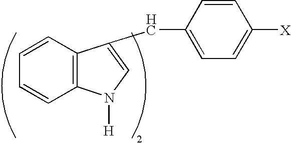
wherein X is H, trifluoromethyl, or methoxy.

15. The method of claim 10, wherein the disease is a cancer.

16. The method of claim 15, wherein the cancer is selected from the group consisting of adrenal cortical cancer, anal cancer, bile duct cancer, bone cancer, bone metastasis, brain cancer, cervical cancer, non-Hodgkin's lymphoma, rectum cancer, esophageal cancer, eye cancer, gallbladder cancer, gastrointestinal carcinoid tumor, gestational trophoblastic disease, Hodgkin's disease, Kaposi's sarcoma, kidney cancer, laryngeal and hypopharyngeal cancer, leukemia, liver cancer, lung cancer, lung carcinoid tumor, malignant mesothelioma, metastatic cancer, multiple myeloma, myelodysplastic syndrome, nasal cavity and paranasal cancer, nasopharyngeal cancer, neuroblastoma, oral cavity and oropharyngeal cancer, osteosarcoma, ovarian cancer, pancreatic cancer, prostate cancer, breast cancer, colon cancer, bladder cancer, penile cancer, pituitary cancer, retinoblastoma, salivary gland cancer, sarcoma, skin cancer, stomach cancer, testicular cancer, thymus cancer, thyroid cancer, uterine sarcoma, vaginal cancer, vulva cancer, and Wilm's tumor.

17. The method of claim 10, wherein the mammal is a human.

18. A method for inducing apoptosis in a cell comprising contacting the cell with an effective dose of an agonist of Nur77.

19. The method of claim 18, wherein the agonist of Nur77 is a diindolylmethane.

20. The method of claim 19, wherein the agonist of Nur77 is a methlylene-substituted diindolylmethane.

21. The method of claim 20, wherein the agonist of Nur77 is a 1,1-bis (3′-indolyl)-1-(p-substituted phenyl) methane.

22. The method of claim 21, wherein the agonist of Nur77 has the chemical formula: 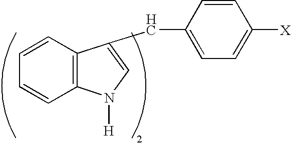
wherein X is H, trifluoromethyl, or methoxy.

23. The method of claim 18, wherein the cell is a cancer cell.

24. The method of claim 23, wherein the cancer cell is selected from the group consisting of adrenal cortical cancer cell, anal cancer cell, bile duct cancer cell, bone cancer cell, bone metastasis cell, brain cancer cell, cervical cancer cell, non-Hodgkin's lymphoma cell, rectum cancer cell, esophageal cancer cell, eye cancer cell, gallbladder cancer cell, gastrointestinal carcinoid tumor cell, gestational trophoblastic disease cell, Hodgkin's disease cell, Kaposi's sarcoma cell, kidney cancer cell, laryngeal and hypopharyngeal cancer cell, leukemia cell, liver cancer cell, lung cancer cell, lung carcinoid tumor cell, malignant mesothelioma cell, metastatic cancer cell, multiple myeloma cell, myelodysplastic syndrome cell, nasal cavity and paranasal cancer cell, nasopharyngeal cancer cell, neuroblastoma cell, oral cavity and oropharyngeal cancer cell, osteosarcoma cell, ovarian cancer cell, pancreatic cancer cell, prostate cancer cell, breast cancer cell, colon cancer cell, bladder cancer cell, penile cancer cell, pituitary cancer cell, retinoblastoma cell, salivary gland cancer cell, sarcoma cell, skin cancer cell, stomach cancer cell, testicular cancer cell, thymus cancer cell, thyroid cancer cell, uterine sarcoma cell, vaginal cancer cell, vulva cancer cell, and Wilm's tumor cell.

25. The method of claim 18, wherein the cell is a human or non-human mammalian cell.

26. The method of claim 25, wherein the cell is in vivo or in vitro.


SEQUENCE LISTING (6 sequences)

SEQ ID NO: 1 - DNA, 28 bp, Artificial sequence
  NBRE oligonucleotides used in gel mobility shift assays
gatcctcgtgcgaaaaggtcaagcgcta

SEQ ID NO: 2 - DNA, 33 bp, Artificial sequence
  NurRE oligonucleotides used in gel mobility shift assays
gatcctagtgatatttacctccaaatgccagga

SEQ ID NO: 3 - DNA, 23 bp, Artificial sequence
  TRAIL forward primer used for RT-PCR
cgtgtactttaccaacgagctga

SEQ ID NO: 4 - DNA, 21 bp, Artificial sequence
  TRAIL reverse primer used for RT-PCR
acggagttgccacttgacttg

SEQ ID NO: 5 - DNA, 21 bp, Artificial sequence
  TBP forward primer used for RT-PCR
tgcacaggagccaagagtgaa

SEQ ID NO: 6 - DNA, 20 bp, Artificial sequence
  TBP reverse primer used for RT-PCR
cacatcacagctccccacca